(19) United States
(12) Patent Application Publication
(10) Pub. No.: US 2007/0281330 A1
(43) Pub. Date: Dec. 6, 2007

(54) Method for Examining the Activity of Ion Channels

(76) Inventors: Andreas Ebneth, Pinneberg (DE); Rainer Netzer, Hamburg (DE); Ulrike Hahn, Vogelsen (DE)

Correspondence Address: JACOBSON HOLMAN PLLC, 400 SEVENTH STREET N.W., SUITE 600, WASHINGTON, DC, 20004, US

(21) Appl. No.: 10/586,329

(22) PCT Filed: Jan. 14, 2005
(86) PCT No.: PCT/EP05/00301

§ 371 Date: Jun. 4, 2007

Related U.S. Application Data
(60) Provisional application No. 60/536,514, filed on Jan. 15, 2004.

(51) Int. Cl. C12Q 1/02

(52) U.S. Cl. 435/29

(57) ABSTRACT

The present invention relates to a method for examining the activity of ion channels, comprising the following steps: providing a sample comprising ion channels; and determining a value of a measuring parameter as an indicator of the activity of the ion channels; characterized in that said determining of the value of the measuring parameter is performed at a temperature of ≦ about 10° C.

The present invention relates to a method for examining the activity of ion channels.

The membranes of living cells serve a wide variety of functions which are of great importance to the integrity and activity of cells and tissues. The delimitation and regulation of the cell contents, exchange of matter and transmission of signals are examples of such functions. Charged molecules and inorganic ions (such as Na⁺, K⁺, Ca²⁺ and Cl⁻ ions) cannot cross membranes by simple diffusion through the lipid bilayer, but require specific transport systems of the membrane. In particular, such transport systems comprise ion channels, of which a wide variety are very well characterized, inter alia, in terms of their biochemical and electrophysiological properties, not least because of their immense importance to various clinical pictures. Such ion channels can be opened and closed selectively, so that ions cannot constantly flow through. The net flow of individual ions is determined by factors like the permeability for the respective ions, the concentration gradient of the ion and the electric potential difference between the two sides of the membrane. Generally, ion channel types which respond to a change of electric potential (voltage-dependent ion channels) are distinguished from those which respond to specific messengers, so-called transmitters. Further, ion pumps are known which provide for active ion transport against the electrochemical gradient with consumption of energy. In this way, characteristic differences in the ion concentrations between the intracellular and extracellular spaces are generated or maintained. One important example is the so-called sodium-potassium pump which enables a coupled transport of sodium and potassium with consumption of ATP as energy source.

A wide variety of clinical pictures are known which are treated with drugs by selectively influencing the activity of ion channels. These include, inter alia, anti-arrhythmic agents, i.e., agents for the treatment of cardiac arrhythmia, which are subdivided into different classes according to their electrophysiological mechanisms of action. Thus, for example, the calcium antagonist verapamil acts through the blocking of calcium channels, whereas members of the potassium antagonists, such as amiodarone and sotalol, cause a selective extension of the duration of action potentials by blocking potassium channels. Other clinical pictures which can be influenced by activating or blocking ion channels include, for example, a large number of CNS diseases (e.g. epilepsy, pain, stroke, migraine), auto-immune diseases, cancer or diabetes.

Within the scope of research on pharmaceutically active substances, it is desirable to have test methods by which the influence of a potentially pharmacologically active substance on such ion channels can be exactly monitored. This is of importance, on the one hand, in the development of substances whose mechanism of action is based on a selective affection of ion channels, and on the other hand, in the evaluation of potential side effects, i.e., the undesirable affection of ion channel activities by the putative drugs.

The membrane potential of living cells is predominantly determined by the intracellular and extracellular sodium, potassium and chloride ion concentrations. For example, if one examines the influence of the blocking of a potassium channel on the resting membrane potential of living cells, the conductivity for potassium through the membrane is essentially changed according to the Goldmann-Hodgkin-Katz equation, which has an effect on the membrane potential. Cells can respond to this change, inter alia, by changing the conductivity for other ions through the membrane to reduce or even prevent a net influence of the potassium channel blocking on the membrane potential. This can be done through the activation of pump systems in the cells which actively transport ions.

Now, if one monitors the membrane potential of living cells under the influence of a potential or known pharmacologically active substance in a test method, there is a risk that the potential value measured is biased due to the counter-regulation mechanisms described. This biasing can even be so high that an affection of the membrane potential may not be recognizable when the signal-to-noise ratio is unfavorably high.

Thus, it is the object of the present invention to provide a test method for examining the activity of ion channels which minimizes the above mentioned interferences.

This object is achieved by a method having the features of independent claim 1. The further claims dependent on claim 1 relate to preferred embodiments of the present invention. Additional claims are directed to the use of specific means to conduct the method according to the present invention.

The present invention relates to a method for examining the activity of ion channels, comprising the following steps:
providing a sample comprising ion channels; and
establishing a value of a measuring parameter as an indicator of the activity of the ion channels;
   characterized in that said establishing of the value of the measuring parameter is performed at a temperature which is significantly lower than the usual room temperature, especially ≦ about 10° C.

The present invention is based on the recognition that, by decreasing the temperature, it is possible to deprive the cells of the possibility of influencing the membrane potential by the activation of the above mentioned pump systems. Test methods described in the prior art, e.g., for the evaluation of potassium channel blockers, are typically performed at body temperature, i.e., 37° C., or at room temperature. If the cells are deprived of the possibility of influencing the membrane potential by the activation of the above mentioned pump systems (or if such possibility is at least significantly reduced) by reducing the temperature, the cells cannot respond as effectively to a change of the membrane potential by blocking potassium or sodium channels as they can at 37° C. or at room temperature.

According to the present invention, it is preferred to perform a determination of the measuring parameter at temperatures of about ≦10° C., especially at about ≦5° C. Particularly preferred are temperatures of about ≦2° C. The lower temperature limit is preferably 0° C. Since the samples (e.g. cell samples) to be examined are typically contained in isotonic buffer solutions, it is also possible, in principle, to perform the measurement slightly below 0° C., typically down to −2° C. or −4° C.

Typically, the measurements are conducted utilizing cells which contain the ion channels. Such cells might be wild-type cells or might be genetically modified cells which e.g. over-express the ion channel under study. However, it is also possible to conduct the measurements according to the present invention on tissues or on cell organelles such as mitochondria. In a further embodiment, it is also possible to prepare membrane fractions or vesicles containing the ion channels of interest and to conduct the measurements according to the present invention on such preparations. Membrane fractions and vesicles might be prepared according to standard methods known in the art of cell fractionation, typically by a lysis of cell pellets obtained from centrifugation of cells in buffer comprising protease inhibitors. The lysate is then typically centrifuged again to pellet debris and organelles. The resulting supernatant typically is spun to collect membranes. Afterwards, the resultant pellet is re-suspended in appropriate buffer to conduct the measurements according to the present invention. If desired, vesicles with uniform size can be obtained through brief sonication. As a further alternative, one might conduct the measurements according to the present invention on ion channels embedded into artificial membranes.

Ion channels to be studied according to the present invention are typically associated with membranes, such as the plasma membrane of cells, the membrane of cell organelles, vesicular membrane or even an artificial membrane. Typically, human or animal cells and cell organelles are used as such, or vesicles or membrane fractions are prepared from such cells and cell organelles. As taught by the present invention, such ion channels are examined by determining a value of a measuring parameter as an indicator of the activity of the ion channels at a decreased temperature compared to room temperature or body temperature. Preferably, the measuring parameter is the membrane potential of the cell, cell organelle, vesicle or artificial membrane, or a measure thereof. In a further embodiment, the measuring parameter might be an ion concentration or a measure thereof. The concentrations of ions such as potassium, sodium, chloride and/or calcium might be studied. Preferably, the measuring parameter is an extracellular and/or intracellular ion concentration of the ions mentioned above, or a measure thereof. In addition, rubidium assays, in particular non-radioactive Rb⁺ flux assays, have found widespread application in drug discovery and development for the analysis of potassium and nonselective cation channels in the pharmaceutical industry. Rubidium is an ideal tracer for potassium channels. It has the same size and same charge as potassium and is permeable to potassium channels. It is a preferred target to detect as it is not present in biological systems. Consequently, it does not add residual background noise to the experimental set-up.

In particular, it is preferred that the value of the measuring parameter be established before, during and/or after the addition of a test substance which (potentially) influences the activity of the ion channels under study. In particular, the activity of a transmitter-dependant ion channel can be examined. However, it is also possible to establish the activity of a voltage-sensitive ion channel. This may be, in particular, the activity of a potassium channel, sodium channel, chloride channel or calcium channel.

Said establishing of a measure of the ion concentration or the membrane potential may be effected, for example, by fluorescence methods, radioactive methods or atomic absorption spectroscopy.

Many different means may be used to measure the membrane potential or the ion concentration, including but not limited to ion-sensitive or voltage-sensitive dyes, chelating agents and rubidium.

For example, the fluorescence emission of a voltage-sensitive fluorescent dye, especially the dye Dibac₄(3), can serve as a measure of the membrane potential, as set forth in more detail below. However, it may also be preferred to measure the ion concentration of rubidium (as an exchange ion), especially of non-radioactive rubidium. Further, the ion concentration of calcium, for example, may be measured by means of chelating agents.

The determination of the membrane potential, which is to be performed at the low temperatures according to the invention, may be effected, in particular, by means of per se known fluorescence assays using commercially available fluorescence readers (e.g., FLIPR of Molecular Devices), confocal fluorescence microscopes or flow-cytometric apparatus. Typically, potential-sensitive fluorescence dyes, such as the commercially available distribution dye bis(1,3-dibutylbarbituric acid)trimethine oxonol (Dibac₄(3)), can be employed. Dibac₄(3) is a dye of the bisoxonol type whose distribution in the cytosol is increased when the membrane is depolarized. This process is accompanied by an increase of fluorescence intensity. Thus, if the dye enters the cells upon depolarization of the resting membrane potential of the cells, for example, due to the blocking of voltage-dependent potassium channels, an increase of the fluorescence activity can be detected. In addition, the quantum yield of this oxonol derivative is advantageously favored by the decreased temperatures. Thus, the accompanying increase of signal intensity can cause a still improved signal-to-noise ratio, in addition to the above described inhibition of the cellular pump systems.

Another advantage relates to the stability of the signal to be read out. When the test is performed at 37° C. or at room temperature, the fluorescence signals (e.g., Dibac₄(3) fluorescence) initially caused by the addition of a channel blocker (such as a potassium channel blocker) to cells can be restored to the initial condition after a short transient increase, inter alia, due to the activity of endogenous ion pumps. This means that the time slot for measuring an effect of substances on the ion channels is narrow, so that an on-line measurement is to be made within a very short time slot (typically less than 2 min) after the addition of the test substance, which puts high demands on the measuring device. If the test is performed at decreased temperatures, such a drop of the initially caused signal is not observed, or only so to a much reduced extent. This has the advantage that the measurement of an effect of a substance on an ion channel to be examined can be made also after several hours of incubation. This in turn significantly facilitates the screening of a large number of substances and thus increases the through-put. Many ion channels become accessible to screening only due to a conversion of a transient signal into a stable read-out parameter.

Apart from the above mentioned Dibac₄(3) dye, other dyes of the bis-barbituric acid oxonol type may be used such as DiSBAC₂(3) or DiBAC₄(5) which are commercially available (e.g. by the supplier Molecular Probes). Also other oxonol dyes such as bis-isoxazolone oxonol dyes (e.g. Oxonol V and Oxonol VI) may be applied. Further voltage-sensitive indicators include carbocyanine derivatives (e.g. indo-, thia-, and oxa-carbocyanines as well as iodide derivatives of carbocyanines), rhodamine dyes, merocyanine 540 and styryl dyes. Among the styryl dyes, one might apply dyes of the aminonaphtylethenylpyridinium type such as di-4-ANEPPS, di-8-ANEPPS, di-2-ANEPEQ, di-8-ANEPPQ, di-12-ANEPPQ or di-1-ANEPIA which are all commercially available (Molecular Probes). Also RH-dyes of this or other suppliers may be used such as RH 414, RH 421, RH 795 or RH 237. As ion-sensitive indicators one might use well-known and commercially available calcium indicators (e.g. fluo-calcium indicators, fura indicators such as benzofuranyl derivatives, indo indicators such as indol derivatives, Calcium Green™ such as CAS No 186501-28-0 or Oregon Green™ such as CAS No 172646-19-4; Molecular Probes) or sodium/potassium indicators (e.g. SBFI, PBFI, Sodium Green Na⁺ indicator, CoroNa Green Na⁺ indicator, CoroNa Red Na⁺ indicator; Molecular Probes). This and other suppliers also provide chelating agents for the conductance of ion channel experiments. Further, one might also apply the FLIPR Membrane Potential Assay Kit (Molecular Devices) used in the experiments set forth below.

In the following, the present invention is illustrated in various experimental examples.

### EXAMPLE 1

### HERG Channel

CHO cells stably transfected with the voltage-dependent potassium channel HERG were trypsinized and centrifuged off. Thereafter, the cells were taken up in 1× buffer (10 mM HEPES, pH 7.3, 140 mM Na⁺, 2 mM K⁺, 1 mM MgCl₂, 2 mM CaCl₂) with 4 μM DiBAC₄(3) (Molecular Probes) and added at 2·10⁴/well in 50 μl to a 384 well microtitration plate having a transparent bottom to which the following substances had been preliminarily added: 5 μl of buffer (2 mM K⁺), 5 μl of buffer+300 mM K⁺, and 5 μl of buffer+10 μM E4031/2 mM K⁺.

Dibac₄(3) served as a voltage-dependent fluorescence dye which enters the cell through the cell membrane upon depolarization of the cell membrane (e.g., caused by increasing the extracellular potassium concentration or by blocking potassium channels), where it binds to intracellular proteins and membranes, which results in an increase of fluorescence.

In the above described wells, the potassium concentration was brought to 2 mM (zero check) and 30 mM (control depolarization) by adding a suitable stock solution. As an antagonist of the HERG potassium channel, E4031 was added at a potassium concentration of 2 mM.

After 150 minutes of incubation on ice and subsequent incubation for 30 minutes at room temperature, the read-out was performed on a commercially available fluorescence reader (Fluostar, bmg) at an excitation wavelength of 485 nm and an emission wavelength of 525 nm.

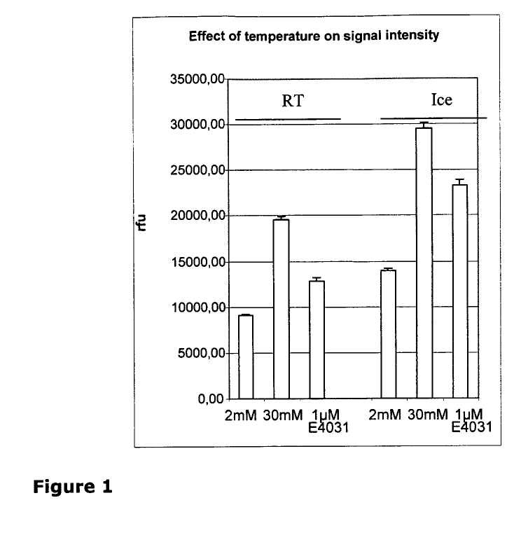

The results of the above described test method are summarized in Table 1 below and in FIG. 1. It can be seen that the increase in fluorescence signal caused by blocking the HERG potassium channel by the antagonist E4031 is significantly stronger for incubation on ice as compared to room temperature. The enhancement of the signal increases from 41.76% at room temperature to 66.62% on ice.

TABLE 1

| | | | |
|---|---|---|---|
| | | | Signal |
| Mean value | | | enhancement |
| of rfu* | Std. dev.** | Error [%] | [%]*** |

150 min of incubation on ice plus 30 min of subsequent incubation at
room temperature

| | | | | |
|---|---|---|---|---|
| 2 mM K⁺ | 9037.67 | 211.27 | 2.34 | |
| 30 mM K⁺ | 19496.33 | 376.26 | 1.93 | 115.72 |
| 1 μM | 12811.67 | 350.54 | 2.74 | 41.76 |
| E4031 | | | | |

150 min of incubation on ice

| | | | | |
|---|---|---|---|---|
| 2 mM K⁺ | 13997.67 | 217.94 | 1.56 | |
| 30 mM K⁺ | 29495.67 | 648.56 | 2.20 | 110.72 |
| 1 μM | 23323.00 | 567.56 | 2.43 | 66.62 |
| E4031 | | | | |

*rfu: relative fluorescence intensity
**Std. dev.: standard deviation
***Enhancement of the established fluorescence signal as compared to the zero check (fluorescence signal at 2 mM K⁺)

In addition to the example 1 set forth above, further experiments were conducted to show the generic applicability of the method according to the present invention to other ion channels than the HERG-channel. Experiments were carried out utilizing the following ion channels: Kv1.1 (example 2), Kv1.5 (example 3), KCNQ1/KCNE1 (examples 4 and 6), Kv1.3 (examples 5 and 7) and SCN5a (example 8). The following general description of chemicals, cell culture, membrane potential assays and data analysis relates to all of these additional examples 2-8.

Chemicals

Chemicals were purchased from Sigma, Merck and Calbiochem. DiBAC4(3) was from Molecular Probes. FLIPR Membrane Potential Assay Kit (FMP) was from Molecular Devices. Toxins were purchased from Alomone Labs.

Cell Culture

CHO cell lines and HEK293 cell lines (wild type and stably transfected with the respective ion channels) were maintained and established at Evotec OAI AG (Hamburg, Germany). CHO cell lines were grown in 75 cm²-flasks (Falcon) in 12 ml MEM ALPHA Medium (Gibco Invitrogen). HEK293 cell lines were grown in DMEM (Gibco Invitrogen). Both media were supplemented with 10% (v/v) fetal calf serum, 1% (v/v) L-glutamine solution (Gibco Invitrogen) and G-418 (geneticine) (800 μg/ml) and grown at 37° C. and 5% CO₂. Cells were split according to standard cell culture protocols.

For performance of the membrane potential assay described in more detail below, the cells were either seeded (50 μl/well) into 384-well microplates (Falcon, Becton Dickinson), incubated for overnight at 37° C. and 5% CO₂ in the above described media before subjecting them to the assay or directly seeded onto the plates at a density of 2·10⁴ cells/well in assay buffer (see below).

Membrane Potential Assay

In the present examples, this fluorescence-based assay makes use of fluorescent dyes (DiBAC4(3) and FMP dye) which either move into or out of the cells depending on the cells' membrane potential. Upon depolarisation of the cells, the dyes enter the cell and bind to intracellular hydrophobic sites, which in turn lead to an increased fluorescence intensity of the dyes.

The culture medium was removed from the cells grown in the microtiter plates. The cells were subsequently covered with 10 μl HEPES buffer (10 mM HEPES, pH 7.2, 5 mM K⁺, 140 mM Na⁺, 5 mM Glucose, 1 mM MgCl₂, 2 mM CaCl₂). DiBAC4(3) was used at a 4 μM concentration and FMP was used close to the manufacturer's instructions, respectively. Compounds to be tested in the assay were dissolved in DMSO at appropriate stock concentrations. Toxins were dissolved in PBS (phosphate-buffered saline buffer: 137 mM NaCl, 2.7 mM KCl, 4.3 mM Na₂HPO₄.7H₂O, 1.4 mM KH₂PO₄; pH˜7.3) supplemented with 1 mg/ml bovine serum albumin.

When adherent cells were used, the medium was removed and replaced by the respective assay buffer. The compounds or toxins were added subsequently. In case suspension cells were used, the compounds and toxins were added to the plate and the cells that were re-suspended in assay buffer after trypsinization were added into the wells. The 30 mM potassium added to the cells (see below) serves as a depolarization-positive control in all experiments.

DiBAC4(3) fluorescence signals were measured after the indicated time of incubation (mostly after several hours—see below) and temperature of incubation (in between 0° C. and 4° C.) using a BMG FLUOstar fluorescence reader (BMG Labtechnologies) or Safire reader (Tecan): excitation wavelength 485 nm (12 nm bandwidth), emission wavelength 520 nm (35 nm bandwidth) in case of DiBAC4(3) or the Safire-reader with excitation wavelength of 540 nm (2.5 nm bandwidth) and emission wavelength of 555 nm (2.5 nm bandwidth) in case of FMP. Fluorescence was measured from below.

Data Analysis

The relative changes in fluorescence intensity were calculated as follows:

$$[ \% ] increase = 100 \times \frac{( {F}_{c} - {F}_{0} )}{{F}_{0}}$$

F₀: fluorescence intensity [rfu] of CHO- or HEK293-cells expressing an ion channel under standard conditions (zero control, 2 mM or 5 mM potassium)
F_{c}: fluorescence intensity [rfu] of CHO- or HEK293-ion channel expressing cells in the presence of compounds/toxins

### EXAMPLE 2

### Kv 1.1

The following Table 2 shows the results obtained after incubation of the cells for 95 minutes at 4° C. in the presence of 5 mM potassium (zero control), 30 mM potassium (positive control), and 100 nM of the toxins delta-DTX and DTX-K (both purchased from Alomone Labs, Israel):

TABLE 2

| | | | | [%] |
|---|---|---|---|---|
| | [rfu] | Std. dev. | Error [%] | increase |
| HEK1.1; | 31570.13 | 221.10 | 0.70 | 40.94 |
| 30 mM K⁺ | | | | |
| HEK1.1; | 22399.53 | 414.47 | 1.85 | |
| 5 mM K⁺ | | | | |
| HEK1.1; | 30732.47 | 233.29 | 0.76 | 37.20 |
| deltaDTx | | | | |
| HEK1.1; | 31707.80 | 861.33 | 2.72 | 41.56 |
| DTx-K | | | | |

“Rfu” denotes the relative fluorescence intensity.
“Std. dev.” denotes the standard deviation.
“[%] increase” denotes the increase of the fluorescence signal of the cells in the presence of the respective ion channel blockers compared to the fluorescence signal of the cells under control conditions (see formula above)

The following Table 3 shows the results obtained after incubation of the same plate cells for 95 minutes at 4° C. followed by an additional incubation for 35 minutes at 37° C. in the presence of 5 mM potassium (zero control), 30 mM potassium (positive control), and 100 nM of the toxins delta-DTX and DTX-K (both purchased from Alomone Labs, Israel).

TABLE 3

| | | | | [%] |
|---|---|---|---|---|
| | [rfu] | Sdt. dev. | Error [%] | increase |
| HEK1.1; | 21754.33 | 117.32 | 0.54 | 18.73 |
| 30 mM K⁺ | | | | |
| HEK1.1; | 18322.87 | 371.40 | 2.03 | |
| 5 mM K⁺ | | | | |
| HEK1.1; | 20750.80 | 109.49 | 0.53 | 13.25 |
| deltaDTx | | | | |
| HEK1.1; | 21109.80 | 450.98 | 2.14 | 15.21 |
| DTx-K | | | | |

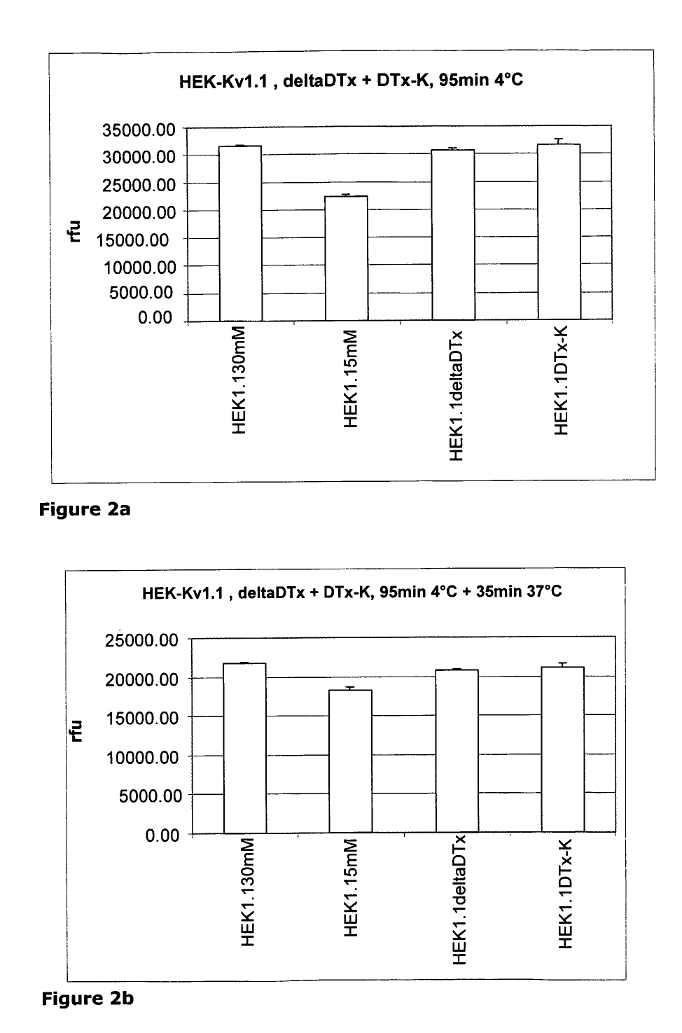

Incubating the cells in the presence of the inhibitors leads to a significantly more pronounced signal when the cells are incubated at low temperatures as taught by the present invention (compare [%] increase in case of the toxins deltaDTx and DTX-K: increase from approx. 14% at 37° C. to ˜40% at 4° C.). See also FIGS. 2a and 2b.

### EXAMPLE 3

### Kv 1.5

Semliki Forest Viruses carrying the Kv1.5 potassium channel were prepared according to the procedure described in Lundstrom et al., 1994, Eur. J. Biochem. 224:917-921 and were stored at −20° C. Before use, the virus was activated with alpha-Chymotrypsinogen (0.2 mg/ml) for 15 min at room temperature. Activation of virus was stopped by addition of 1/50 volume aprotinin (20 mg/ml). CHO-cells were seeded the day before in 25 cm²-flasks, medium (see above under Cell Culture) was removed and replaced with 1 ml fresh medium and 450 μl of the virus-containing supernatant as well as 20 μl HEPES-buffer (10 mM N-[2-Hydroxyethyl] piperazine-N′-[2-ethanesulfonic acid] pH 6.9). After incubation for 90 minutes another 5 ml fresh medium was added and cells were incubated for 12 hours.

The following days, the cells were harvested by trypsinization, washed once in PBS (phosphate-buffered saline buffer: 137 mM NaCl, 2.7 mM KCl, 4.3 mM Na₂HPO₄.7H₂O, 1.4 mM KH₂PO₄; pH˜7.3) and re-suspended in assay buffer (10 mM HEPES, pH 7.2, 5 mM K⁺, 140 mM Na⁺, 5 mM Glucose, 1 mM MgCl₂, 2 mM CaCl₂) containing FMP before adding to 384-wells loaded with a standard Kv1.5 blocker. Plates were incubated for 15 minutes at room temperature and subsequently cooled down to 1° C. for 30 minutes. As can be seen in Table 4, the relative increase of the relative fluorescence units of the cells in the presence of the Kv1.5-specific blocker is significantly higher when cells are incubated at low temperature as taught by the present invention (63% relative increase compared to 26%).

TABLE 4

| | | | | Increase |
|---|---|---|---|---|
| | [rfu] | Std. dev. | Error [%] | [%] |
| 15 min at room temperature | | | | |
| Zero control | 28275.86 | 718.22 | 2.54 | |
| 30 mM K⁺ | 34496.79 | 1011.96 | 2.93 | 22.00 |
| 10 μM compound | 35711.50 | 1849.73 | 5.18 | 26.30 |
| 30 min 1° C. | | | | |
| Zero control | 29256.36 | 1412.06 | 4.83 | |
| 30 mM K⁺ | 47019.86 | 1579.57 | 3.36 | 37.78 |
| 10 μM compound | 47737.07 | 2159.66 | 4.52 | 63.17 |

### EXAMPLE 4

### KCNQ1/KCNE1

The following Table 5 shows the results obtained after incubation of the cells for 15 minutes at room temperature and additional 180 minutes in the presence of 2 mM potassium (zero control), 30 mM potassium (positive control), and 10 μM of a standard antagonist according to the procedure described above using suspension cells and the FMP-dye. Again, the relative signal increase is significantly higher when cells are incubated at low temperature as taught by the present invention (109% relative increase compared to 24% increase when incubated at room temperature).

TABLE 5

| | | | | Increase |
|---|---|---|---|---|
| | [rfu] | Std. dev. | Error [%] | [%] |
| 30 min room temperature | | | | |
| Zero control | 6037.80 | 370.07 | 6.13 | |
| 30 mM K⁺ | 12067.03 | 752.27 | 6.23 | 99.86 |
| 10 μM compound | 7517.19 | 359.33 | 4.78 | 24.50 |
| 180 min 1° C. | | | | |
| Zero control | 9968.25 | 749.07 | 7.51 | |
| 30 mM K⁺ | 25157.61 | 1237.40 | 4.92 | 152.38 |
| 10 μM compound | 20893.36 | 864.68 | 4.14 | 109.60 |

### EXAMPLE 5

### Kv 1.3

The following Table 6 shows the results obtained after incubation of the cells for 10 minutes at room temperature and additional 110 minutes at 1° C. in the presence of 5 mM potassium (zero control), 3 mM potassium (positive control), and 100 nM of Margatoxin (Alomone labs) according to the procedure described above using suspension cells and the FMP-dye. The relative signal is significantly higher when cells are incubated at low temperature as taught by the present invention (68% relative increase compared to 3% increase when incubated for 10 minutes at room temperature).

TABLE 6

| | | | | Increase |
|---|---|---|---|---|
| | [rfu] | Std. dev. | Error [%] | [%] |
| 10 min room temperature | | | | |
| Zero control | 16994.63 | 491.21 | 2.89 | |
| 30 mM K⁺ | 27062.25 | 460.50 | 1.70 | 23.91 |
| 100 nM Margatoxin | 22479.50 | 968.56 | 4.31 | 2.93 |
| 110 min 1° C. | | | | |
| Zero control | 28251.75 | 1523.77 | 5.39 | |
| 30 mM K⁺ | 60872.63 | 3569.17 | 5.86 | 115.46 |
| 100 nM Margatoxin | 47474.00 | 2678.98 | 5.64 | 68.04 |

### EXAMPLE 6

### Screening Application on KCNQ1/KCNE1

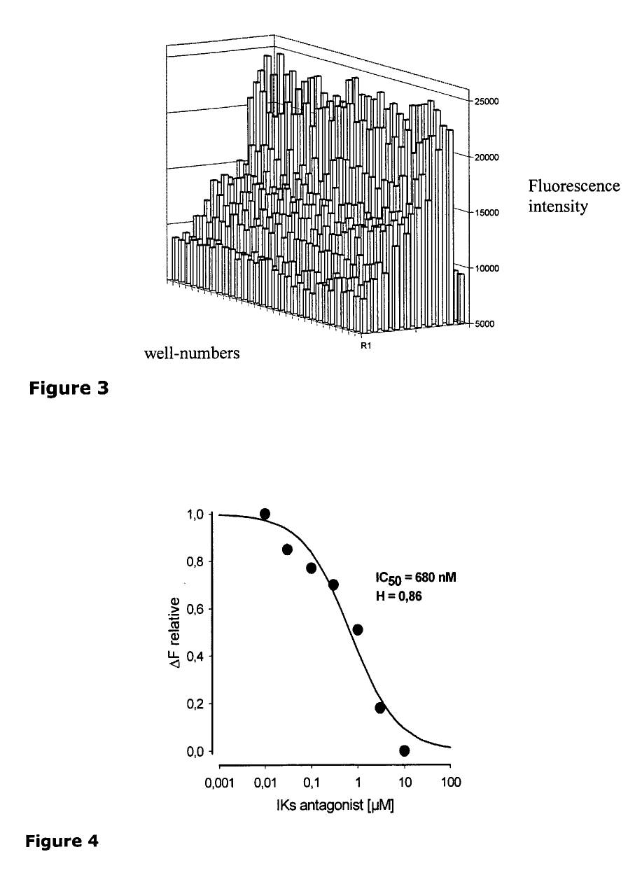

The examination of ion channels according to the present invention provides for an increased signal-to-noise ratio compared to methods known in the art. This results in statistical data sufficient for high-throughput screening campaigns. Investigations of control compounds on the KCNQ1/KCNE1 potassium channel (Iks) under HTS-conditions in 1536-well plates resulted in z′-factors of >0,6. Examples of the investigations of an Iks antagonist in 1536-well plates applying the present invention are depicted in FIGS. 3 and 4. FIG. 3 reflects the increase of fluorescence with increasing concentrations of the antagonist. In the first two and the last two rows of the figure, the control values are shown (without compound). The fluorescence intensity in these control rows is significantly lower than the one in rows showing the fluorescence intensity of wells to which compounds have been applied. From the third column, results of increasing concentrations from 0.01 to 10 μM of the compound are shown. With increasing concentrations of the compound, the fluorescence intensity increases. An example for a respective concentration/response relation is shown in FIG. 4.

### EXAMPLE 7

### Screening Application on Kv 1.3

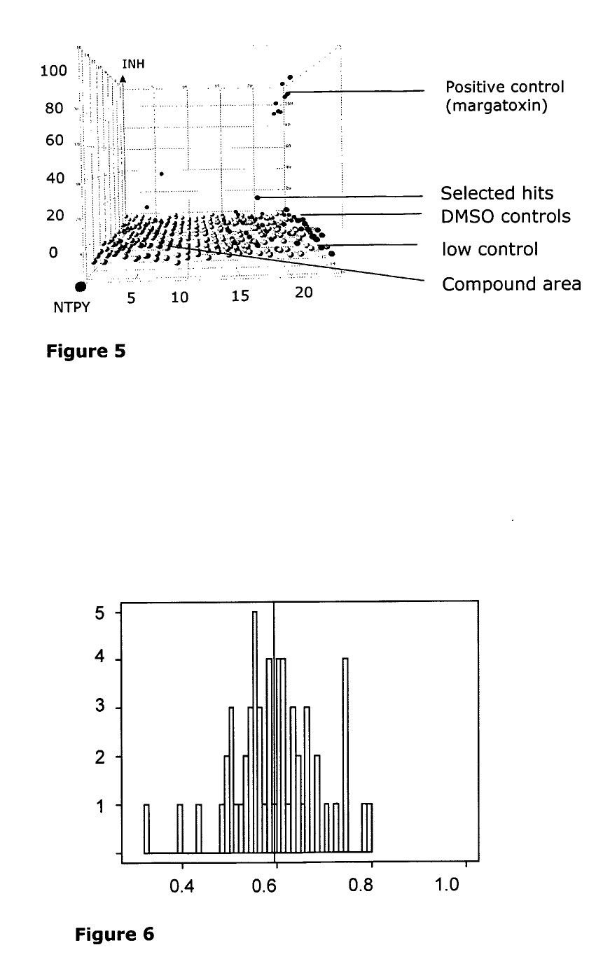

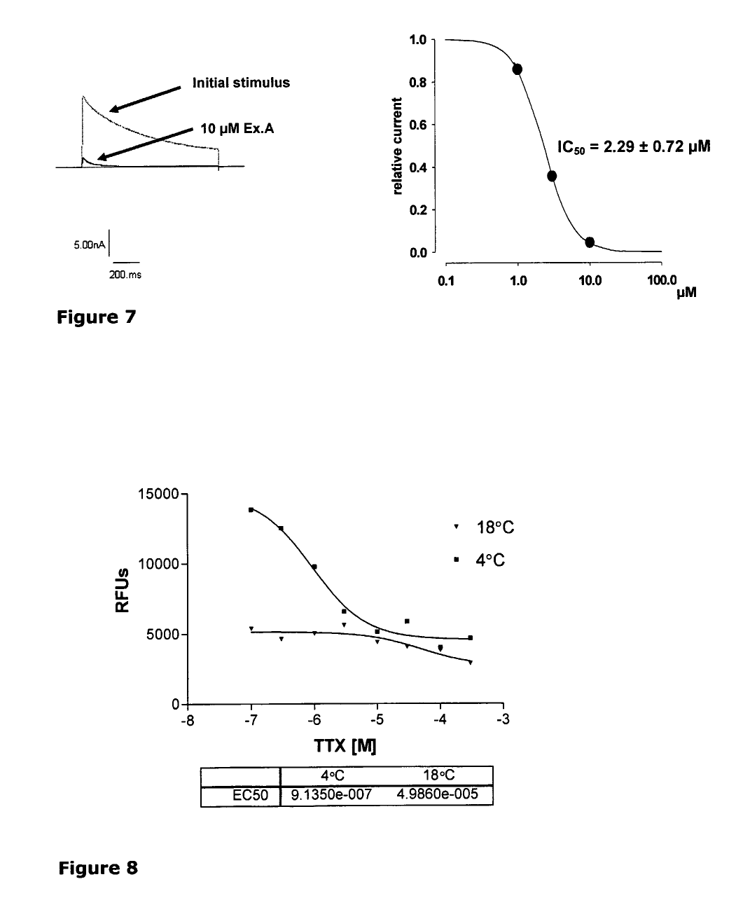

The invention was used for screens of larger numbers of compounds. In the present example, the measurements of 25.000 compounds on a Kv1.3 potassium channel were performed in 384-well plates. The investigations of the compounds resulted in an increase of the fluorescence when antagonists of the Kv1.3 channel were applied to the cells. Investigations of compounds expressing the Kv1.3 potassium channel are depicted in FIG. 5 (margenta: positive control margatoxin; blue: low controls; green: DMSO negative controls; grey: compound area; red: selected hits). The calculation of the mean z′ of the investigations of 25.000 compounds on the Kv1.3 resulted in a value of approximately 0,6, as shown in FIG. 6. Compounds detected as hits by applying the method according to the present invention, were confirmed as Kv1.3 antagonists using electrophysiological methods (patch-clamp technique) as shown in FIG. 7.

### EXAMPLE 8

### SCN5a

In the present example, the sodium channel SCN5a expressed in a mammalian cell line is investigated. Cells were loaded in standard assay buffer using the Molecular Devices kit described above at room temperature for 30 mins. After loading, tetrodotoxin (TTX) was applied to different concentrations and incubated for a further 10 minutes after which Veratradine to an end concentration of 50 μM was added and the plates were incubated on ice or at room temperature for various periods of time.

The following table shows the results after 17 minutes incubation.

TABLE 7

| TTX [μM] | 4° C. [RFUs] | 18° C. [RFUs] |
|---|---|---|
| 3.00E+02 | 4687.0 | 2882.0 |
| 1.00E+02 | 4019.0 | 3816.0 |
| 3.00E+01 | 5873.0 | 4067.0 |
| 1.00E+01 | 5143.0 | 4401.0 |
| 3.00E+00 | 6610.0 | 5620.0 |
| 1.00E+00 | 9772.0 | 5034.0 |
| 3.00E−01 | 12512.0 | 4636.0 |
| 1.00E−01 | 13840.0 | 5379.0 |

The IC50 value was calculated for both incubation conditions and is shown in FIG. 8. Incubation of cells with compounds at low temperature according to the present invention leads to much improved sensitivity of detection as well as signal-to-noise ratio. At low temperature incubation, the reported IC 50 value is in excellent agreement with literature.

1. A method for examining the activity of ion channels, comprising the following steps:
providing a sample comprising ion channels; and
determining a value of a measuring parameter as an indicator of the activity of the ion channels, the measuring parameter being a membrane potential, a measure of a membrane potential, an ion concentration, or a measure of an ion concentration;
characterised in that said determining of the value of the measuring parameter is performed at a temperature of ≦ about 10° C. by fluorescence methods, radioactive methods or atomic absorption spectroscopy.

2. The method according to claim 1, characterized in that said determining of the value of the measuring parameter is performed at a temperature of ≦ about 5° C., especially ≦ about 2° C.

3. The method according to claim 1, characterized in that said determining of the value of the measuring parameter is performed at a temperature of from about 10° C. to −4° C. especially from about 5° C. to −4° C., more preferably from about 5° C. to 0° C., even more preferably from about 2° C. to 0° C.

4. The method according to claim 1, characterized in that the sample comprises one or more cells or cell organelles which have ion channels, in particular human or animal cells or cell organelles.

5. The method according to claim 1, characterized in that the sample comprises one or more vesicles which have ion channels.

6. The method according to claim 1, characterized in that the sample comprises membrane bound ion channels, in particular ion channels embedded into a membrane of cells, cell organelles, vesicles or embedded into an artificial membrane.

7. The method according to claim 1, characterized in that said measuring parameter is the membrane potential of a cell, cell organelle or vesicle, or a measure of said membrane potential.

8. The method according to claim 1, characterized in that the measuring parameter is an extracellular, intracellular, extravesicular and/or intravesicular ion concentration or a measure thereof.

9. The method according to claim 1, characterized in that the value of said measuring parameter is determined before, during and/or after the addition of a test substance which potentially influences the activity of the ion channels.

10. The method according to claim 1, characterized in that the activity of a transmitter-dependent ion channel is examined.

11. The method according to claim 1, characterized in that the activity of a voltage-sensitive ion channel is examined.

12. The method according to claim 1, characterized in that the activity of a potassium channel, chloride channel, sodium channel or calcium channel is examined.

13. The method according to claim 1 characterized in that an optical response of (i) a carbocyanine derivative, in particular a thia-, indo-, or oxa-carbocyanine or an iodide derivative of a carbocyanine, (ii) a rhodamine dye, (iii) an oxonol dye, (iv) merocyanine 540, or (v) a styryl dye serves as a measure of the membrane potential.

14. The method according to claim 1, characterized in that the fluorescence emission of a voltage-sensitive fluorescent dye, preferably a DiBAC dye, more preferably the dye Dibac₄(3), serves as a measure of the membrane potential.

15. The method according to claim 1, characterized in that the ion concentration of rubidium, especially of non-radioactive rubidium, is determined as an indicator of the activity of the ion channels.

16. The method according to claim 1, characterized in that the ion concentration, especially the ion concentration of calcium, is measured by means of chelating agents.

17. The method according to claim 1, characterized in that the values of several measuring parameters are determined.

18. The method according to claim 1 for use in the research on pharmaceutically active substances, especially in the medium- or high-throughput screening of potentially or established active pharmaceutical substances, in particular the identification of potentially active pharmaceutical substances or the determination of side effects of potentially or established active pharmaceutical substances.

19. The method according to claim 1 for use in the agricultural research, especially in the research on agrochemicals as e.g. insectizids.

20. Use of a voltage-sensitive or ion-sensitive indicator for the conductance of the method according to claim 1.

21. Use according to claim 20 wherein the ion-sensitive indicator is a calcium indicator, in particular a fluo-calcium indicator, a fura indicator, an indo indicator, Calcium Green™, or Oregon Green™.

22. Use according to claim 20 wherein the ion-sensitive indicator is a sodium or potassium indicator, preferably a fluorescent sodium or potassium indicator, in particular SBFI, PBFI, Sodium Green Na⁺ indicator, CoroNa Green Na⁺ indicator, or CoroNa Red Na⁺ indicator.

23. Use according to claim 20 wherein the voltage-sensitive indicator is a carbocyanine derivative, in particular an indo-, thia-, or oxa- carbocyanine or a iodide derivative of a carbocyanine; a rhodamine dye; an oxonol dye; merocyanine 540; or a styryl dye.

24. Use according to claim 23 wherein the oxonol dye is a bis-isoxazolone oxonol dye or a bis-barbituric acid oxonol (DiBAC) dye, in particular DiBAC₄(3), DiSBAC₂(3) or DiBAC₄(5).

25. Use according to claim 23 wherein the styryl dye is an ANEP (AminoNaphthylEthenylPyridinium) dye, in particular di-4-ANEPPS, di-8-ANEPPS, di-2-ANEPEQ, di-8-ANEPPQ, di-12-ANEPPQ, di-1-ANEPIA, or a dialkylaminophenylpolyenylpyridinium dye (RH dye), in particular RH 414, RH 421, RH 795 or RH 237.

26. Use of a chelating agent for the conductance of the method according to claim 1.

27. Use of rubidium, in particular non-radioactive rubidium, for the conductance of the method according to claim 1.

28. Use of an atomic absorption spectrometer, a flow cytometer, a fluorescence microscope or fluorescence plate reader for the conductance of the method according to claim 1.

29. Use of an atomic absorption spectrometer, a flow cytometer, a fluorescence microcope or fluorescence plate reader for applying a voltage-sensitive or ion-sensitive indicator according to claim 20.

30. Use of an atomic absorption spectrometer, a flow cytometer, a fluorescence microcope or fluorescence plate reader for applying a chelating agent according to claim 26.

31. Use of an atomic absorption spectrometer, a flow cytometer, a fluorescence microcope or fluorescence plate reader for applying rubidium according to claim 27.
